(19) United States
(12) Patent Application Publication
(10) Pub. No.: US 2016/0106932 A1
(43) Pub. Date: Apr. 21, 2016

(54) DEVICE AND METHOD FOR HINDERING FORMATION OF LIPOHYPERTROPHY

(71) Applicant: Elijah Bruce Zapzalka, Wayzata, MN (US)

(72) Inventor: Elijah Bruce Zapzalka, Wayzata, MN (US)

(21) Appl. No.: 14/840,720

(22) Filed: Aug. 31, 2015

Related U.S. Application Data
(60) Provisional application No. 62/064,764, filed on Oct. 16, 2014.

(51) Int. Cl. A61M 5/42; A61M 5/31

(52) U.S. Cl. CPC A61M 5/427; A61M 5/31; A61M 5/003

(57) ABSTRACT

A device for rotating sites of subcutaneous injections into external tissue is provided. The device comprises a pad having an interlaced structure. The device further comprises a liquid disposed within the interlaced structure. The liquid is configured to leave a visible imprint onto the external tissue upon contact of the pad with the external tissue.

### CROSS-REFERENCE TO RELATED APPLICATION

The present application is based on and claims the benefit of U.S. provisional patent application Ser. No. 62/064,764, filed Oct. 16, 2014, the content of which is hereby incorporated by reference in its entirety.

### BACKGROUND

The discussion below is merely provided for general background information and is not intended to be used as an aid in determining the scope of the claimed subject matter.

A person's body operates using three sources of energy: protein, fat, and carbohydrates. Sugar is a carbohydrate, and although many different types of sugars exist, a type of sugar called glucose has a principal role in fueling the body, as glucose provides a source of instant energy. As such, glucose is one of the main type of sugars that can be found in the bloodstream of the person. Glucose comes from foods the person eats and is one of the major sources of energy needed to fuel the functions of the body. After the person eats a meal, the body breaks down the eaten foods into glucose and other nutrients, all of which are absorbed into the bloodstream. The level of glucose in the blood rises after the meal and subsequently triggers the pancreas to make a hormone called insulin and release the insulin into the bloodstream.

Insulin is a chemical substance made in one part of the body that travels through the bloodstream to a distant part of the body to control the level of glucose in the blood. Insulin is said to act like a key to open a cell in the body so that glucose can enter the cell. If glucose is unable to enter the cell, glucose cannot provide energy to the body. Insulin enables fat and muscle to form, stores glucose for use when fuel is not coming in, and blocks the breakdown of protein. With the normal source of insulin in the bloodstream, the body keeps the level of glucose steady at approximately 60 to 100 mg/dl at all times. But, when there is insufficient amount of insulin or when insulin is not working effectively, glucose starts to rise in the bloodstream. When glucose rises, usually above 180 mg/dl, and the kidneys—as filters for the blood—are unable to extract the glucose before it enters the urine, the loss of glucose into the urine leads to diabetes mellitus.

Diabetes mellitus (also known as diabetes) refers to a group of metabolic conditions during which a person experiences abnormally high levels of glucose in his or her bloodstream. This may be due to the person's inadequate production of insulin and/or cells of a body's inability to properly respond to injected hormones, such as insulin. A person having high blood sugar levels will typically also experience polyuria (frequent urination), polydipsia (increasing thirst), and polyphagia (increasing hunger).

Insulin is a hormone produced in the pancreas, which is located behind the stomach, by special cells, called beta cells. To move glucose into the cells, insulin is needed. There, the insulin is stored and used later for energy. When a person has diabetes, that person either lacks islet cells that produce insulin or that person's fat, liver, and muscle cells become insulin resistant, that is, they do not respond correctly to insulin. As a result, blood sugar does not get into these cells and cannot be stored for energy. When sugar is unable to enter the cells, hyperglycemia results. Hyperglycemia is a condition where an excessively high level of sugar builds up in the blood.

According to the World Health Organization, there are several types of recognized classes of diabetes, including Insulin Dependent Diabetes Mellitus (IDDM) or Type I Diabetes, Non-Insulin Dependent Diabetes Mellitus (NIDDM) or Type II Diabetes, and Gestational Diabetes. Type 1 Diabetes—initially called Juvenile onset diabetes—affects mostly adolescents and young adults. Type 1 diabetes is an autoimmune condition in which an immune system of a person attacks cells that produce insulin. This results in little or no amount of insulin provided by the body meaning this individual is no longer self-sustaining and cannot live without the use of artificial insulin. Type 1 Diabetes is caused by a sudden failure of the pancreas to produce insulin. Type 1 Diabetes is an acute disease, presenting with thirst, polyuria (passing large amount of urine), diuresis (increased or excessive urine production) and weight loss. Type 2 Diabetes is the most prevalent type of diabetes, and is found mostly in adults and the elderly. Type 2 Diabetes develops gradually over a long period of time and is characterized by insufficient insulin, deficient insulin in the blood or the inability of the body to utilize the insulin resent (insulin resistance). Gestational diabetes is a condition that develops during pregnancy, usually halfway through, in which blood sugar levels are too high. With gestational diabetes, there are rarely symptoms. However, if present, symptoms are mild. Although not life-threatening to the pregnant woman, failure to treat gestational diabetes can bring extra glucose to the baby and thus cause the baby to put on extra weight. The blood sugar levels usually return to normal after delivery of the baby. However, the baby is more prone to having periods of hypoglycemia (low blood sugar) during the first few days of life.

To successfully cope living with any of the types of diabetes, a person with diabetes has to ensure that insulin enters his or her bloodstream. This can be accomplished through injections into the fatty layers of the body, such as the arms, thighs, or abdomen. For some people, injections are needed on a daily basis. However, injecting a needle in or near the same site of injection within a short amount of time of each injection can lead to lypohypertrophy.

Lypohypertrophy is a term used to describe a medical condition where a lump, swelling, or thickened skin forms under the skin of humans caused by accumulation of extra fat at a site of numerous subcutaneous injections of hormones used to regulate carbohydrate and fat metabolism in the body of human, such as, for example, insulin. A typical injection site affected by lypohypertrophy extends several inches across and is rounded. The site of formation of the lump may be painful and could change the timing or completeness of the injected hormone. The affected injection site often contains adipose tissue (the tissue inside the site of injection) that is more firm than ordinary subcutaneous fat and, although connective, stands loose. Injecting insulin into tissue that is more firm or loose than ordinary subcutaneous fatty issue can affect a rate at which the injected insulin hormone is absorbed by the body, leading to inaccurate and erratic blood glucose results.

Lypohypertrophy will gradually disappear as long as injections in the same area are avoided. However, that process may take several months.

### SUMMARY

This Summary and the Abstract herein are provided to introduce a selection of concepts in a simplified form that are further described below in the Detailed Description. This Summary and the Abstract are not intended to identify key features or essential features of the claimed subject matter, nor are they intended to be used as an aid in determining the scope of the claimed subject matter. The claimed subject matter is not limited to implementations that solve any or all disadvantages noted in the Background.

An aspect of the present disclosure relates to a device for rotating sites of subcutaneous injections into external tissue. The device includes a pad that comprises an interlaced structure. The device further includes a liquid disposed within the interlaced structure. The liquid is configured to leave a visible imprint onto the external tissue upon contact of the pad with the external tissue.

Another aspect of the present disclosure relates to a device for rotating sites of subdermal injections of fluid into external tissue. The device includes a pad that comprises at least two meshed components having the same shape. The paid is formed by joining the two meshed components together. The device further includes a liquid disposed within the pad. The liquid is configured to leave a visible imprint onto the external tissue upon contact of the pad with the external tissue.

Another aspect of the present disclosure relates to a method for rotating sites of subcutaneous injections into external tissue. The method comprises inserting a pad at a base of a needle. The pad is configured to have an interlaced structure, allowing the needle to be fed through the pad. The method further comprises injecting a substance using the needle into external tissue, where the substance is present in a barrel securely attached to the needle. The method further comprises forming a contact between the pad and the external tissue. The method further comprises leaving a visible imprint on the external tissue using a liquid disposed within the interlaced structure based upon the contact.

These and various other features and advantages that characterize the claimed embodiments will become apparent upon reading the following detailed description and upon reviewing the associated drawings.

### BRIEF DESCRIPTION OF THE DRAWINGS

FIG. 1 is a front perspective view of a device for rotating sites of subcutaneous injections into external tissue.

FIGS. 2A-2E are front views of a syringe end.

FIG. 3 is a side view of a syringe pen.

FIG. 4 is an exploded view of the syringe pen.

FIG. 5 is an exploded view of a syringe hub.

FIG. 6 is a perspective view of a syringe pen that is subcutaneously injected into the external tissue.

The figures now have been illustrated and will be described in clear detail enabling one of ordinary skill to make and use the present invention. Modifications can be made without departing from the spirit and scope of the invention.

### DETAILED DESCRIPTION OF ILLUSTRATIVE EMBODIMENTS

To avoid or minimize the formation of lypohypertrophy or similar medical conditions, sites of numerous subcutaneous and subdermal injections should be rotated among areas of a person's body. The areas of the body that are typically rotated include upper outer arms, upper outer thighs, hip, buttocks, and abdomen, among others. Often, rotation charts are provided to aid patients with keeping track of the rotations. However, when more frequent rotation is required, such as in the case of persons with diabetes who have to have multiple injections of insulin into the bloodstream, sometimes on a daily basis, using a rotation chart that precisely denotes where on the arm each injection took place can be challenging.

The present disclosure discloses embodiments of a device and a method directed to hindering formation of lypohypertrophy by rotating sites of subcutaneous injections into external tissue of a person or an animal. The device includes a pad having an interlaced structure. A liquid is disposed within the interlaced structure and is configured to leave a visible imprint onto the external tissue upon contact of the pad with the external tissue.

The term “couple,” “couples” or “coupled” is intended to mean either an indirect or direct connection. Thus, if a first device couples or is coupled to a second device, that connection may be through a direct connection, or through an indirect connection via other devices and connections.

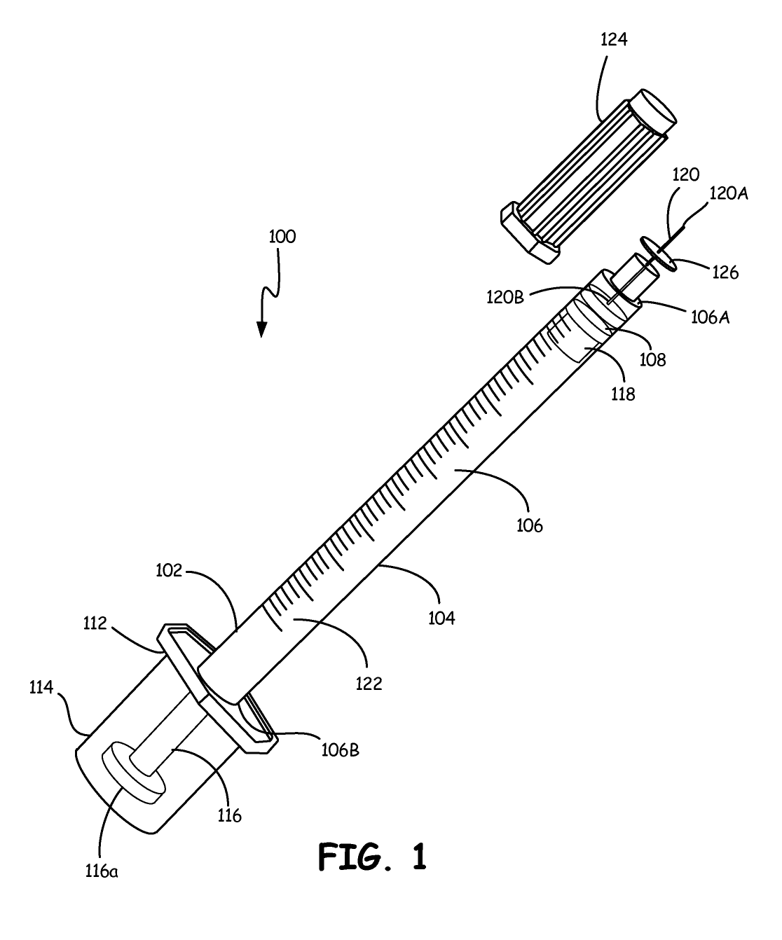

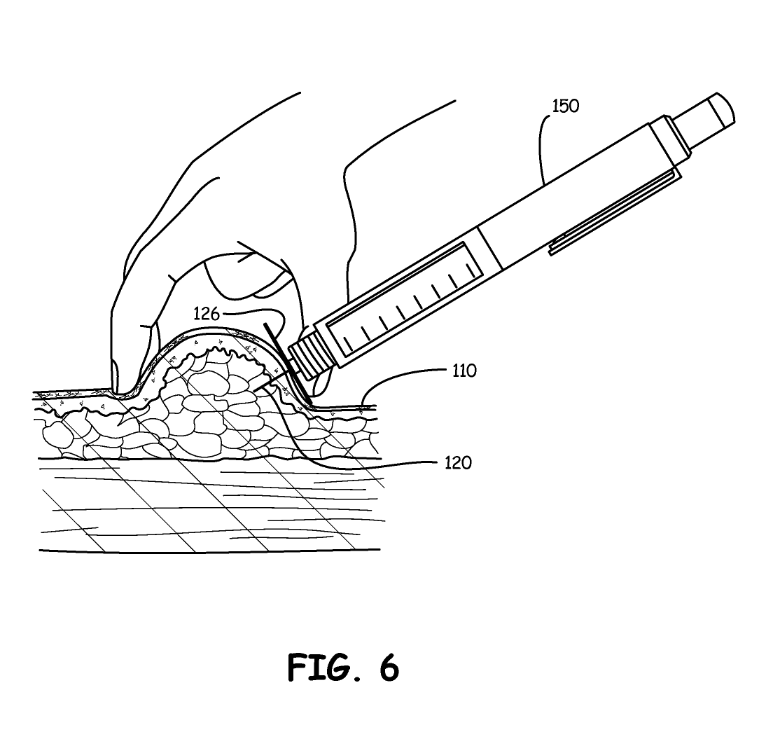

An exemplary embodiment of a device 100 used to hinder the formation of lypohypertrophy is illustrated in FIG. 1. The device 100 includes a syringe 102 having a body 104 with a cavity 106 formed therein. The cavity 106 can be, for example, a barrel having markings used to measure an amount of substance 108 subcutaneously or subdermally expelled and injected into external tissue 110 (shown in FIG. 6) of a human being or an animal. The substance 108 contained in the syringe 102 can be any type of a compound, matter, or medical drug that can be inserted into external tissue 110 subcutaneously to reach the bloodstream intravenously or muscle tissue subdermally. In the case of managing diabetes, the substance can include, but is not limited to, insulin. The substance 108 can include not only clear insulin, but also cloudy insulin mixed with clear insulin.

As shown in FIG. 1, the syringe barrel 106 has long lines and shorter lines. The long lines mark every five units, whereas the shorter lines, which are dispersed in between the long barrel lines, indicate single units. The barrel 106 has wings or flanges 112 used to help hold the syringe and/or administer the substance 108 contained within the syringe barrel 106. In one exemplary embodiment, the device 100 includes an end cap 114 that covers a first end 116A of a plunger 116. Inside the plunger 116, a rubber stopper 118 is provided that moves in a longitudinal direction within the syringe 102 as the plunger 116 is pulled or pushed.

In one embodiment, the device 100 includes a syringe needle 120 removably detached to a first end 106A of the syringe barrel 106 opposite the plunger 116. The needle 120 extends in a direction opposite that of a second end 106B of the syringe barrel 106, where the second end 106B of the barrel 106 is near the syringe end cap 114. The syringe needle 120 has a first end 120A and a second end 120B, where the first end 120A is fluidly coupled to receive fluid 122 from the syringe cavity 106. The syringe needle 120 is configured to extend through a syringe end 126, and is used to subcutaneously insert and administer the substance 122 contained in the barrel 106 into the external tissue 110 intravenously. In one embodiment shown in FIG. 1, the device 100 includes a safety cap 124 covering the needle 120. The safety cap 124 covers a part of the needle 120 that extends from the first end 106A of the syringe barrel 106 and can be removed once the syringe is ready to be used.

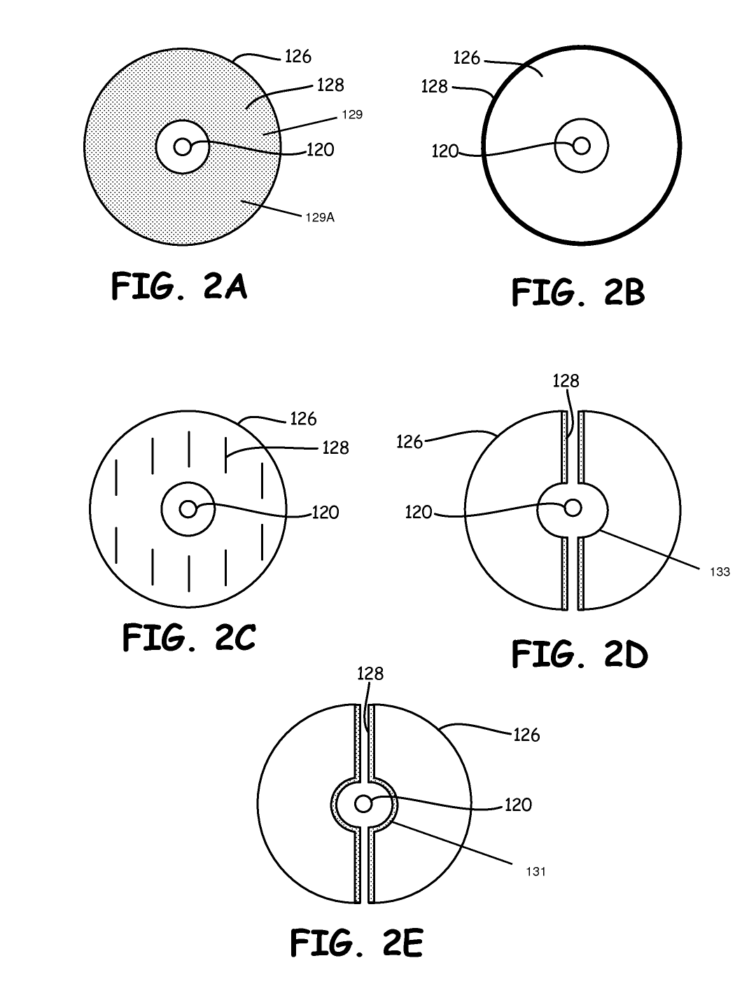

In one illustrative embodiment, the device 100 includes a syringe end 126 having an outer surface 126A with a marking element 128, as shown in FIGS. 2A-2E. In one embodiment shown in FIG. 2A, the marking element 128 can include a pad 129 having an interlaced structure 129A configured to rotate sites of subcutaneous injections into the external tissue. As used herein, the term “interlaced” includes both woven and non-woven classes of materials. In one embodiment, a marking liquid is disposed within the interlaced structure. The marking liquid is configured to leave a visible imprint onto the external tissue upon contact of the pad 129 with the external tissue.

Density of the interlace of the interlaced structure 129A can vary depending upon the size of the needle used to inject the substance. For example, the interlaced structure 129A can be more dense for thinner needles. In one embodiment, the density of the interlace can be used as a fastener that controls adherence of the pad to the needle. In other words, the density of the interlace of the interlaced structure 129A can be used to hold the pad securely in place as the syringe is inverted for application of the injection into the external tissue. It should be noted that in this embodiment, the pad 129 does not include a pre-formed aperture. Rather, the syringe needle moves fibers of the pad 129 apart as it penetrates the surface of the pad 129 and extends therethrough.

In one exemplary embodiment, the marking liquid can be provided after the syringe needle 120 is fed through the pad 129. This configuration lowers the possibility of needle contamination.

In a further embodiment shown in FIGS. 2D-2E, the pad 129 is formed by separate portions joined together, where at least one of the separate portions has an edge for forming an aperture through which the syringe needle 120 can be extended. The separate portions are joined together in a variety of ways, including through the use of an adhesive 131 or bonding. In a non-adhesive configuration shown in FIG. 2D, ultrasonic welding 133 can be used to join the two separate portions together. The size of the aperture can vary in order to accommodate the size of the needle 120 such that the pad 129 will not slide down the needle 120 as the syringe 102 is inverted and as the needle 120 is inserted into the tissue.

It should be noted that embodiments of the present disclosure can be used with a variety of styles of needle tips. Those needle styles include single bevel, multi-bevel and multi-facet needle points, such as Tuohy needle (a hollow hypodermic needle, slightly curved at the end, suitable for inserting epidural catheters); Franseen needle; Whitacre needle; Cournand needle; Mengini needle; Backcut bevel needle; and Dos Santos needle. In one exemplary embodiment, the syringe can have a beveled needle tip (not illustrated) designed for optimum penetration of the septa and prevention of septa coring. For example, a bevel of 17 degrees is considered standard in industry. The tip can be electro-tapered so as to minimize the necessary piercing force and diameter of the injection channel. In another exemplary embodiment, the needle tip has a 90 degree bevel. This type of needle is used when there is a need for precise pipetting, as this design releases drops completely, for maximum reproducibility. In another exemplary embodiment, the needle tip can have an electro-tapered tip polished 90 degree square end in order to help eliminate burns. This type of needle is normally used with polytetrafluoroethylene laminated seals and regular septas. In yet another exemplary embodiment, the needle tip could be equipped with a side opening for dispensing and filling. This design avoids plugging and minimizes septum damage. This needle is typically used for thicker septa.

In one embodiment illustrated in FIG. 2D, an adhesive is provided on a back surface of the pad 129 facing towards the barrel and away from the injection site. The adhesive 131 can be repositionable adhesive and can be used to removably secure the pad 129 to the barrel 106 during injection of the substance into the tissue.

In one embodiment of the present disclosure, to begin the process of inserting insulin subcutaneously into external tissue using a syringe, begin by first injecting air into a syringe cavity formed within a syringe body, where the amount of air is equal to the amount of insulin that will be removed a bottle of insulin. Twist and remove the end cap from the end of the plunger, and then pull the cap off the needle, ensuring to pull straight off in order to avoid damaging the needle. To get X number of units of insulin into the syringe cavity, get X number of units of air into the barrel 106 of the syringe by pulling the plunger until the end of the rubber stopper closest to the needle goes past the X number mark on the barrel 106. An end point of the needle can be placed directly above a center of a top of the bottle. The needle 120 can be pushed straight through the top of the bottle of insulin until the entire needle goes in. The plunger is then slowly pushed down so as to push the air in the syringe into the bottle. With the syringe still into the bottle, turn both the insulin bottle and the syringe upside down such that the bottle is on top of the syringe. To draw insulin into the syringe, slowly and steadily pull down on the plunger until the end of a black rubber stopper closest to the needle is even with the line on the syringe, indicating the dose of insulin desired to be injected into the external tissue. It is important to pull slowly, because pulling too fast may create tiny air bubbles in the syringe which can take the place of the insulin and keep the patient from getting a correct dose. Once a dose without any air bubbles has been drawn up, the syringe and the bottle can be turned back over and the bottle can be placed on a flat surface. Then holding the syringe by the barrel, the needle can be pulled straight out of the insulin bottle and the syringe placed on a flat surface.

As indicated above, to hinder the formation of lypohypertrophy, injection sites are rotated by coupling to the syringe a syringe needle having a first end and a second end, where the first end is fluidly coupled to receive fluid from the cavity and the needle extends through an end with an outer surface with a marking element. For example, the injection site can be moved by a finger's width of each injection.

As shown in FIG. 6, in the case of injecting insulin, it should be noted that the area of tissue where the needle is to be inserted can be pinched up so as to ensure that the needle does not hit any muscle and that the insulin is released into the fatty tissue just beneath the skin. When the needle is fully inserted, slowly push in the plunger to deliver the insulin to the tissue. The pinch should be released as the needle is slowly removed.

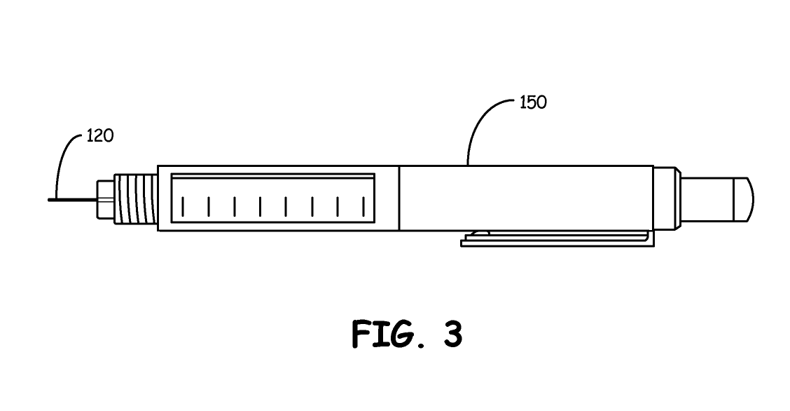

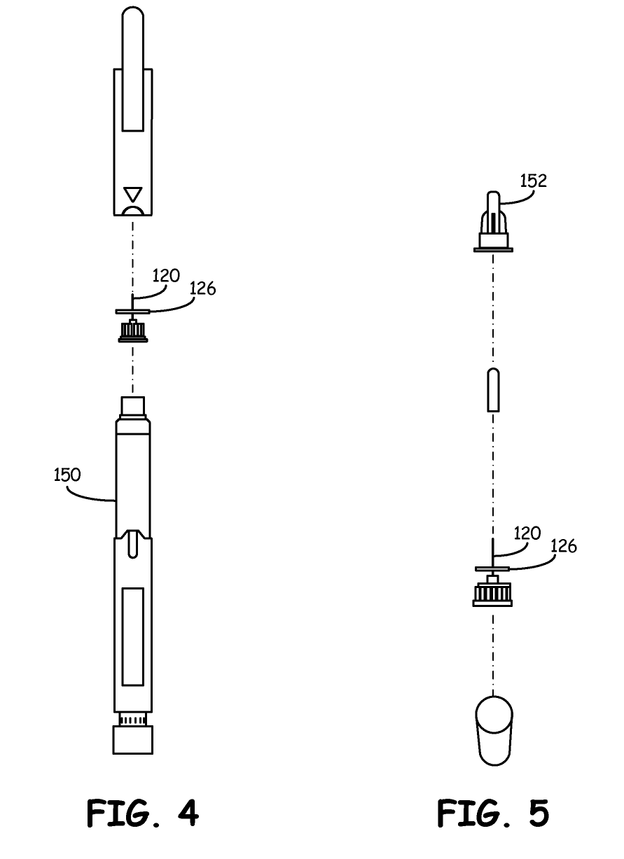

In another exemplary embodiment illustrated in FIGS. 3 and 4, the device includes a syringe pen 150 having a body with a cavity formed therein and a pen needle hub attached to the body. The pen needle has an outer shield, an inner shield, a needle and a paper tab. To attach a new pen needle to the pen 150, the paper tab can be pulled off the pen needle and the tab can be discarded. The new pen needle can then be screwed onto the pen 150. Pull off the outer shield and set it aside, as it will be needed later to remove the pen needle 120 from the pen 150. Pull off the inner shield and discard it. Prime the pen 105 before each injection. Priming refers to the technique of delivering a small amount of insulin through the needle so as to ensure the needle 120 is open and working properly. This step removes air bubbles, which helps to attain an accurate amount of insulin. First select the doze of units by turning the knob. Hold the pen with the needle pointing upward and tap the drug reservoir so that any air bubbles rise up toward the needle. Keeping the needle pointing upward, press the injection needle all the way in and count to five slowly. Check to see if a drop of medication comes out of the needle. If a drop does not form at the end of the pen needle, repeat the priming steps until a drop is seen at the end of the pen needle. Do this for up to two more times. If a drop still does not form, replace the pen needle with a new one. If the pen is new or there are air bubbles in the reservoir, the user may need to repeat the priming sequence several times. The doze window should display the number “0” after the pen has been primed. Adjust the pen for the correct number of units for the doze you need. For insulin pens, the user can set the dozes for one unit steps up to a maximum of 80 units. If more than 80 units are needed, then the user will need to use two or more injections. If the user accidentally turns past the doze, the user can turn the window back down to the correct doze.

Hold the pen 150 in the palm of one hand. Select the injection site—arm, abdomen, outer thighs and upper buttocks. Injection site should be cleaned. If the pen needle is longer than 5 millimeters, then it is suggested that the skin be pinched up so that the insulin will be delivered into the fat under the skin. Press the pen's injection button in all the way. Hold the injection button in that position with the needle in the skin while slowly counting to ten. This is to ensure that the entire dose of insulin medication enters the body. Remove the needle from the injection site. After every injection, the pen needle should be removed from the pen. The pen should be stored without a pen needle attached. This prevents air from entering the insulin, insulin from escaping from the insulin pen, helps prevent leakage of the medication and infection from the use of a non-sterile pen needle. Place the pen onto a flat surface. Guide the pen into the outer shield. Use the outer shield to unscrew the pen needle from the pen. Put the cap on the pen. Always dispose of the pen needle using an approved sharps container. Can use the special opening on top of the sharps container to unscrew the pen needle from the pen. If you do not have access to a sharps container, using a storage and clipping device can be used to store the contaminated needle. Alternatively, can use the outer cover to store the needle until access to an approved sharps container becomes available. Each pen can be used until the medication in the pen is gone or until the expiration date specified by the manufacturer, whichever comes first.

It should be noted that the elements and/or assemblies of the device can be connected with suitable fasteners as necessary to allow the device to be shipped in a disassembled state. Although the subject matter has been described in a language specific to structure features and/or methodological acts, it is to be understood that the subject matter defined in the appended claims is not necessarily limited to the specific features or acts described above as has been determined by the courts. Rather, the specific features and acts described above are disclosed as example forms of implementing the claims. Although the present invention has been described with reference to preferred embodiments, workers skilled in the art will recognize that changes may be made in form and detail without departing from the spirit and scope of the invention.

What is claimed is:

1. A device for rotating sites of subcutaneous injections into external tissue, comprising:
a pad comprising an interlaced structure; and
a liquid disposed within the interlaced structure, wherein the liquid is configured to leave a visible imprint onto the external tissue upon contact of the pad with the external tissue.

2. The device of claim 1, further comprising a needle configured to subcutaneously inject fluid into the external tissue, wherein the needle is removably connected to the pad.

3. The device of claim 2, further comprising a syringe removably connected to the needle and configured to store the fluid for injection into the external tissue.

4. The device of claim 2, further comprising a pen removably connected to the needle and adapted to store the fluid injected into the external tissue.

5. The device of claim 1, wherein the pad comprises an aperture in the interlaced structure to receive a needle.

6. The device of claim 1, further comprising adhesive adapted to connect the pad to the needle.

7. The device of claim 1, wherein the liquid comprises non-toxic ink.

8. A device for rotating sites of subdermal injections of fluid into external tissue, comprising:
a pad comprising at least two meshed components having the same shape, wherein the pad is formed by joining the two meshed components together; and
a liquid disposed within the pad, wherein the liquid is configured to leave a visible imprint onto the external tissue upon contact of the pad with the external tissue.

9. The device of claim 8, wherein the two meshed components are joined using an adhesive.

10. The device of claim 8, wherein the two meshed components are joined using a suitable fastener.

11. The device of claim 8, wherein the two meshed components are coupled to each other to form the pad comprising a whole geometric body.

12. The device of claim 11, wherein the whole geometric body having an elliptical shape.

13. The device of claim 12, wherein the whole geometric body having a round shape.

14. The device of claim 9, wherein the adhesive is on an outer edge of the pad.

15. The device of claim 9, wherein the adhesive is dispersed on one side of the pad.

16. A method for rotating sites of subcutaneous injections into external tissue, comprising:
inserting a pad at a base of a needle, wherein the pad comprises an interlaced structure allowing the needle to be fed through the pad;
injecting a substance using the needle into external tissue, wherein the substance is present in a barrel securely attached to the needle;
forming a contact between the pad and the external tissue;
leaving a visible imprint on the external tissue using a liquid disposed within the interlaced structure based upon the contact.

17. The method of claim 16, wherein the liquid disposed within the interlaced structure comprises non-toxic ink.

18. The method of claim 16, wherein the liquid is contained in a syringe removably connected to the needle.

19. The method of claim 16, wherein the liquid is contained in a pen removably connected to the needle.

20. The method of claim 16, wherein the pad is drawn out of the needle pen upon pressing a release button.
